(19) United States
(12) Patent Application Publication
(10) Pub. No.: US 2011/0152328 A1
(43) Pub. Date: Jun. 23, 2011

(54) Combination Therapy For Glaucoma

(76) Inventors: Scott M. Whitcup, Laguna Hills, CA (US); Robert M. Burk, Laguna Beach, CA (US); David W. Old, Irvine, CA (US); Yariv Donde, Dana Point, CA (US); Wha-Bin Im, Irvine, CA (US); Mark A. Holoboski, Irvine, CA (US)

(21) Appl. No.: 12/988,168

(22) PCT Filed: Apr. 13, 2009
(86) PCT No.: PCT/US2009/040381

§ 371 Date: Mar. 11, 2011

Related U.S. Application Data
(60) Provisional application No. 61/045,492, filed on Apr. 16, 2008.

(51) Int. Cl. A61K 31/426; A61K 31/192; A61K 31/4025; A61K 31/4155; A61K 31/421; A61K 31/427; A61K 31/381; A61P 27/06

(52) U.S. Cl. 514/365; 514/570; 514/422; 514/385; 514/376; 514/372; 514/448

(57) ABSTRACT

Disclosed herein is method of treating glaucoma or ocular hypertension comprising administering a prostaglandin agonist and a second therapeutically active agent to a mammal in need thereof, wherein said second therapeutically active agent is selected from: β-Blockers, Adrenergic Agonists, non-selective adrenergic agonists, α2-selective adrenergic agonists, Carbonic Anhydrase Inhibitors, Cholinergic Agonists, direct acting cholinergic agonists, chlolinesterase inhibitors, Glutamate Antagonists, Ca²⁺ channel blockers, Prostamides, Prostaglandins, Cannabinoids, and combinations thereof. Compositions and medicaments containing a combination of these two active agents are also disclosed.

### CROSS REFERENCE

This application claims priority to U.S. Provisional Application Ser. No. 61/045,492 filed Apr. 16, 2008, which is hereby incorporated by reference in its entirety.

### DESCRIPTION OF THE INVENTION

Disclosed herein is method of treating glaucoma or ocular hypertension comprising administering a prostaglandin agonist and a second therapeutically active agent to a mammal in need thereof, wherein said second therapeutically active agent is selected from: β-Blockers, Adrenergic Agonists, non-selective adrenergic agonists, α₂-selective adrenergic agonists, Carbonic Anhydrase Inhibitors, Cholinergic Agonists, direct acting cholinergic agonists, chlolinesterase inhibitors, Glutamate Antagonists, Ca²⁺ channel blockers, Prostamides, Prostaglandins, Cannabinoids, and combinations thereof.

These compounds may be administered via two compositions, one containing the prostaglandin agonist and one containing the second therapeutically active agent. Alternatively, a composition comprising a combination of the prostaglandin agonist and the second therapeutically active agents is contemplated.

One or more of the compositions above may be in a solid dosage form. Alternatively, one or more compositions may be in the form of an ophthalmically acceptable liquid which is administered topically to one or more eyes of a mammal, including a human being. For example, an ophthalmically acceptable liquid comprising both active agents may be in a single composition that is administered topically to one or more eyes of a mammal, including a human being.

Alternatively, the prostaglandin agonist may be in a topical dosage form and the second therapeutically active agent may be in a solid dosage form. For example, the second therapeutically active agent may be in a solid oral dosage form or may be a solid implant that is installed into the eye of the mammal.

Formulation of useful dosage forms is known in the art. See, for example, U.S. Pat. No. 7,091,231 (columns 23-25), for further information on the subject.

In one embodiment, the prostaglandin agonist and the second therapeutically active agent are administered in separate dosage forms.

In another embodiment, the prostaglandin agonist and the second therapeutically active agent are administered in a single dosage form.

In another embodiment, the prostaglandin agonist is a selective agonist of the prostaglandin EP2 or prostaglandin EP4 receptor.

In another embodiment, the prostaglandin agonist is a compound selected from:

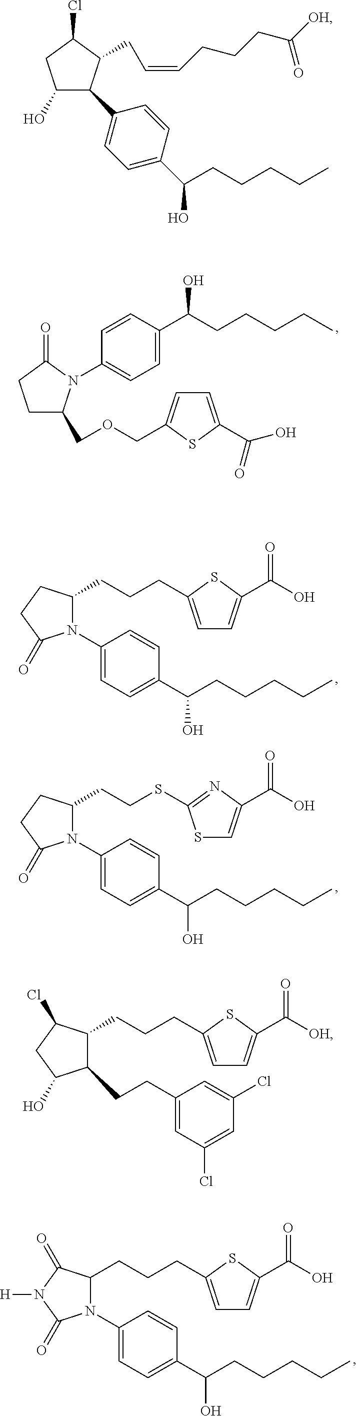

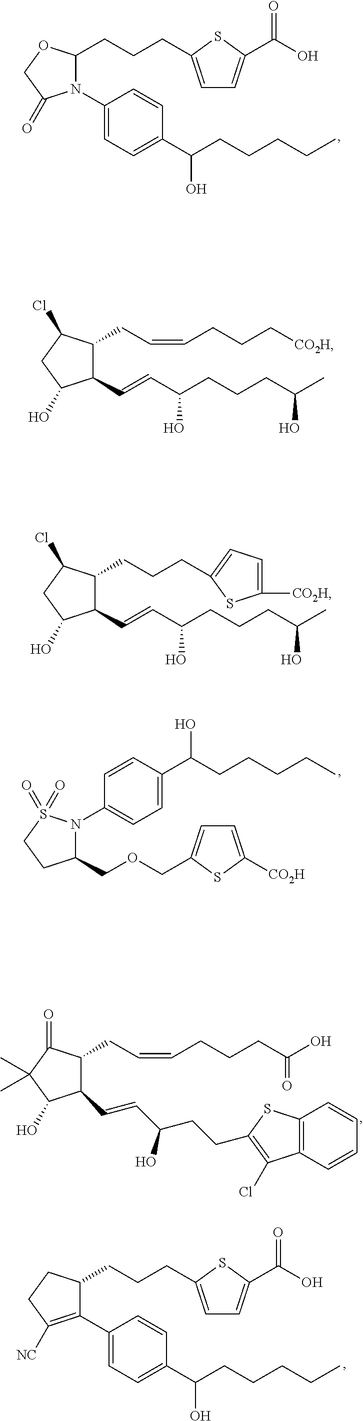

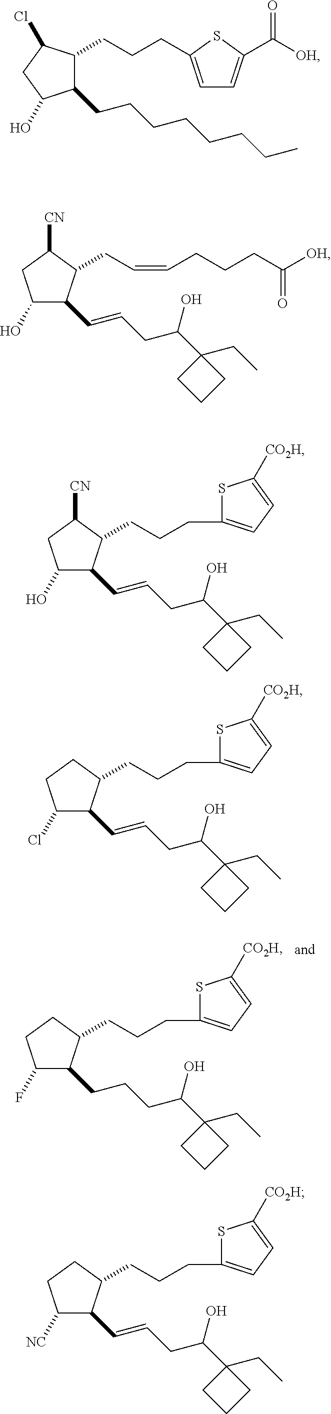

or is a C₁₋₆ alkyl ester, a phenyl ester, a 2-hydroxyethyl ester, or a pharmaceutically acceptable salt thereof.

In another embodiment, the prostaglandin agonist is

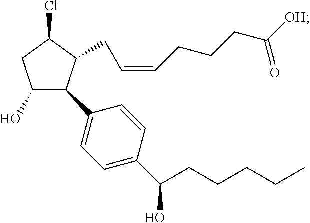

or is a salt, isopropyl ester, or a 2-hydroxyethyl ester thereof.

In another embodiment, wherein the prostaglandin agonist is

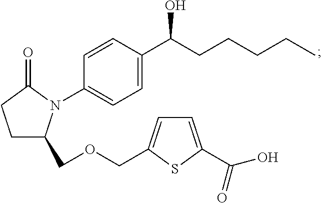

or is a salt, isopropyl ester, or a 2-hydroxyethyl ester thereof.

In another embodiment, wherein the prostaglandin agonist is

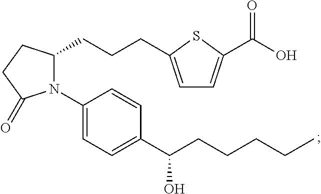

or is a salt, isopropyl ester, or a 2-hydroxyethyl ester thereof.

In another embodiment, wherein the prostaglandin agonist is

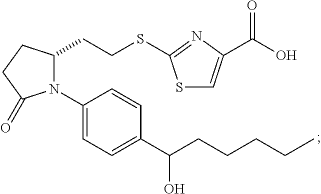

or is a salt, isopropyl ester, or a 2-hydroxyethyl ester thereof.

In another embodiment, wherein the prostaglandin agonist is

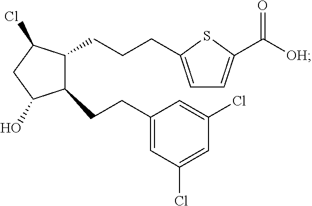

or is a salt, isopropyl ester, or a 2-hydroxyethyl ester thereof.

In another embodiment, wherein the prostaglandin agonist is

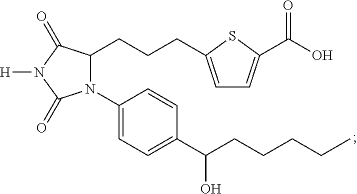

or is a salt, isopropyl ester, or a 2-hydroxyethyl ester thereof.

In another embodiment, wherein the prostaglandin agonist is

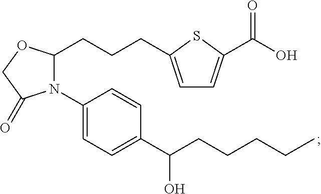

or is a salt, isopropyl ester, or a 2-hydroxyethyl ester thereof.

In another embodiment, wherein the prostaglandin agonist is

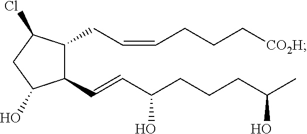

or is a salt, isopropyl ester, or a 2-hydroxyethyl ester thereof.

In another embodiment, wherein the prostaglandin agonist is

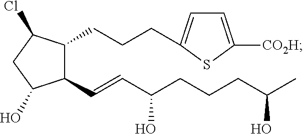

or is a salt, isopropyl ester, or a 2-hydroxyethyl ester thereof.

In another embodiment, wherein the prostaglandin agonist is

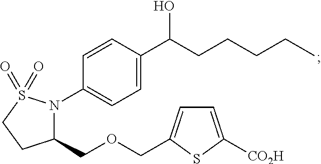

or is a salt, isopropyl ester, or a 2-hydroxyethyl ester thereof.

In another embodiment, wherein the prostaglandin agonist is

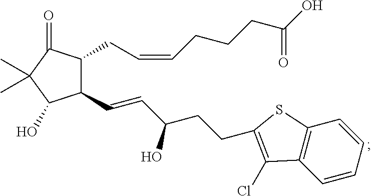

or is a salt, isopropyl ester, or a 2-hydroxyethyl ester thereof.

In another embodiment, wherein the prostaglandin agonist is

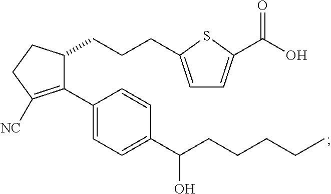

or is a salt, isopropyl ester, or a 2-hydroxyethyl ester thereof.

In another embodiment, wherein the prostaglandin agonist is

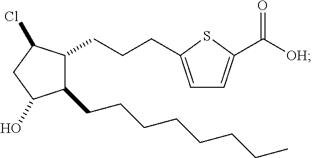

or is a salt, isopropyl ester, or a 2-hydroxyethyl ester thereof.

In another embodiment, wherein the prostaglandin agonist is

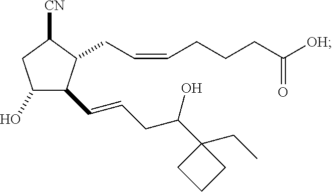

or is a salt, isopropyl ester, or a 2-hydroxyethyl ester thereof.

In another embodiment, wherein the prostaglandin agonist is

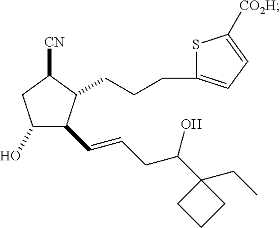

or is a salt, isopropyl ester, or a 2-hydroxyethyl ester thereof.

In another embodiment, wherein the prostaglandin agonist is

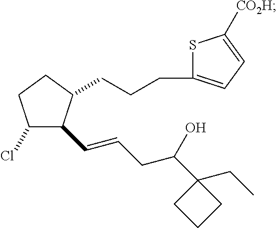

or is a salt, isopropyl ester, or a 2-hydroxyethyl ester thereof.

In another embodiment, wherein the prostaglandin agonist is

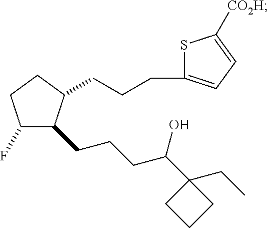

or is a salt, isopropyl ester, or a 2-hydroxyethyl ester thereof.

In another embodiment, wherein the prostaglandin agonist is

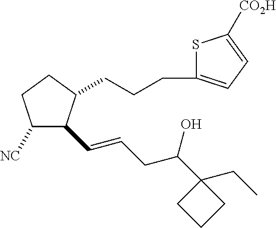

or is a salt, isopropyl ester, or a 2-hydroxyethyl ester thereof.

The carboxylic acids depicted below are examples of useful prostaglandin EP2 or EP4 agonists. These compounds, as well as pharmaceutically acceptable salts of these compounds, C₁₋₆ alkyl esters of these compounds, phenyl esters of these compounds, and 2-hydroxyethyl esters of these compounds, are contemplated for use with a second therapeutically active agent. The names of these compounds are depicted below the structures.

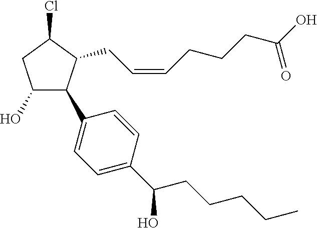

### (Z)-7-((1R,2S,3R,5R)-5-chloro-3-hydroxy-2-(4-((R)-1-hydroxyhexyl)phenyl)cyclopentyl)hept-5-enoic acid

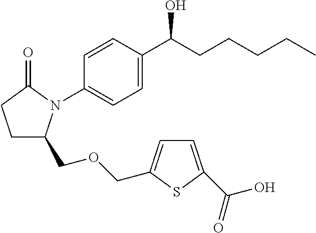

### 5-((((R)-1-(4-((S)-1-hydroxyhexyl)phenyl)-5-oxopyrrolidin-2-yl)methoxy)methyl)thiophene-2-carboxylic acid

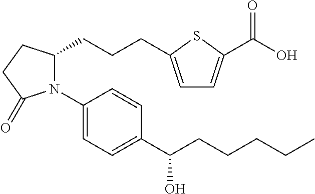

### 5-(3-((S)-1-(4-((S)-1-hydroxyhexyl)phenyl)-5-oxopyrrolidin-2-yl)propyl)thiophene-2-carboxylic acid

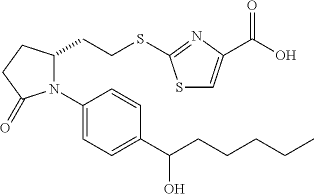

### 2-(2-((2R)-1-(4-(1-hydroxyhexyl)phenyl)-5-oxopyrrolidin-2-yl)ethylthio)thiazole-4-carboxylic acid

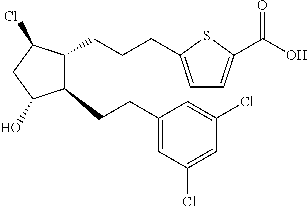

### 5-(3-((1R,2R,3R,5R)-5-chloro-2-(3,5-dichlorophenethyl)-3-hydroxycyclopentyl)propyl)thiophene-2-carboxylic acid

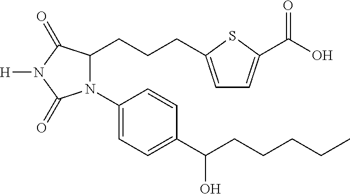

### 5-(3-(3-(4-(1-hydroxyhexyl)phenyl)-2,5-dioxoimidazolidin-4-yl)propyl)thiophene-2-carboxylic acid

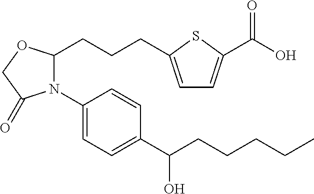

### 5-(3-(3-(4-(1-hydroxyhexyl)phenyl)-4-oxooxazolidin-2-yl)propyl)thiophene-2-carboxylic acid

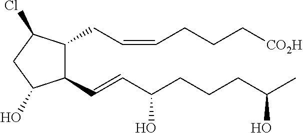

### (Z)-7-((1R,2R,3R,5R)-5-chloro-2-((3S,7R,E)-3,7-dihydroxyoct-1-enyl)-3-hydroxycyclopentyl)hept-5-enoic acid

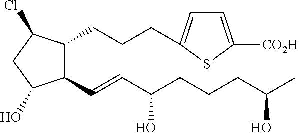

### 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid

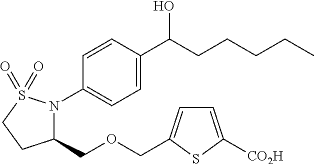

### 2-Thiophenecarboxylic acid, 5-[[[2-[4-(1-hydroxyhexyl)phenyl]-1,1-dioxido-3-isothiazolidinyl]methoxy]methyl]-

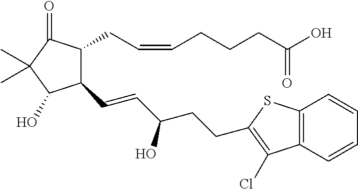

### (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid

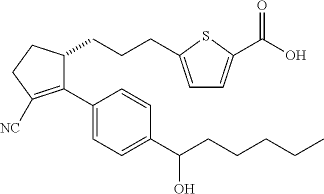

### 5-(3-((1S)-3-cyano-2-(4-(1-hydroxyhexyl)phenyl)cyclopent-2-enyl)propyl)thiophene-2-carboxylic acid

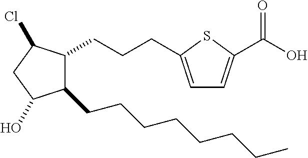

### 5-(3-((1R,2R,3R,5R)-5-chloro-3-hydroxy-2-octylcyclopentyl)propyl)thiophene-2-carboxylic acid

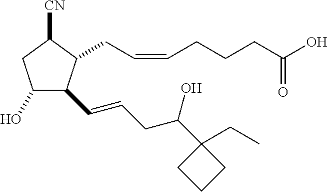

### (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid

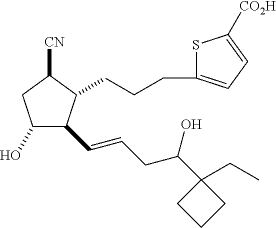

### 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid

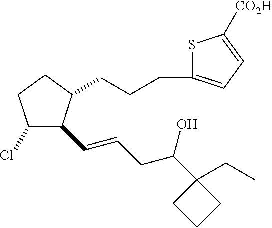

### 5-(3-((1S,2S,3R)-3-chloro-2-((E)-4-(1-ethylcyclobutyl)-4-hydroxybut-1-enyl)cyclopentyl)propyl)thiophene-2-carboxylic acid

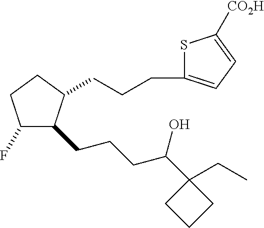

### 5-(3-((1S,2R,3R)-2-(4-(1-ethylcyclobutyl)-4-hydroxybutyl)-3-fluorocyclopentyl)propyl)thiophene-2-carboxylic acid

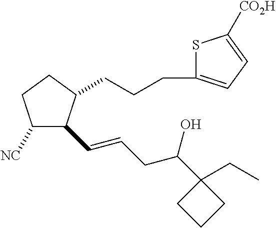

### 5-(3-((1S,2S,3R)-3-cyano-2-((E)-4-(1-ethylcyclobutyl)-4-hydroxybut-1-enyl)cyclopentyl)propyl)thiophene-2-carboxylic acid

These compounds, or pharmaceutically acceptable salts of these compounds, are also contemplated as useful prostaglandin EP2 or EP4 agonists. The names of these compounds are depicted below the structures.

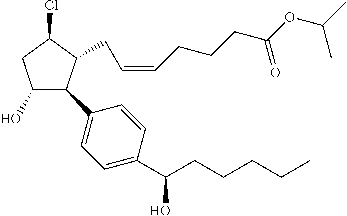

### (Z)-isopropyl 7-((1R,2S,3R,5R)-5-chloro-3-hydroxy-2-(4-((R)-1-hydroxyhexyl)phenyl)cyclopentyl)hept-5-enoate

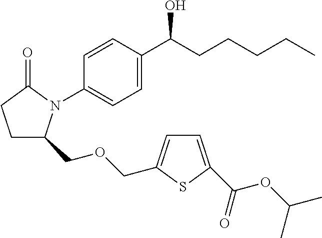

### Isopropyl 5-((((R)-1-(4-((S)-1-hydroxyhexyl)phenyl)-5-oxopyrrolidin-2-yl)methoxy)methyl)thiophene-2-carboxylate

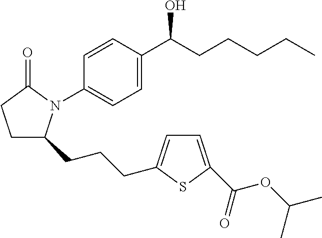

### Isopropyl 5-(3-((S)-1-(4-((S)-1-hydroxyhexyl)phenyl)-5-oxopyrrolidin-2-yl)propyl)thiophene-2-carboxylate

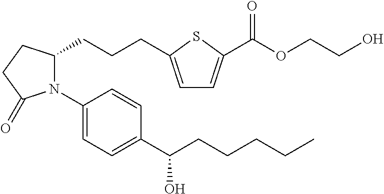

### 2-hydroxyethyl 5-(3-((S)-1-(4-((S)-1-hydroxyhexyl)phenyl)-5-oxopyrrolidin-2-yl)propyl)thiophene-2-carboxylate

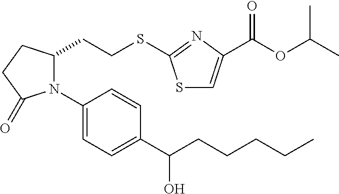

### Isopropyl 2-(2-((2R)-1-(4-(1-hydroxyhexyl)phenyl)-5-oxopyrrolidin-2-yl)ethylthio)thiazole-4-carboxylate

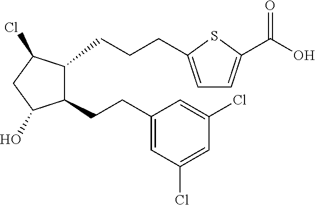

### 5-(3-((1R,2R,3R,5R)-5-chloro-2-(3,5-dichlorophenethyl)-3-hydroxycyclopentyl)propyl)thiophene-2-carboxylic acid

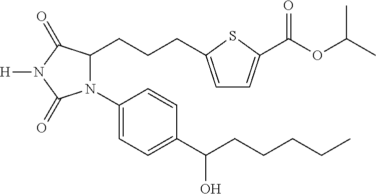

### Isopropyl 5-(3-(3-(4-(1-hydroxyhexyl)phenyl)-2,5-dioxoimidazolidin-4-yl)propyl)thiophene-2-carboxylate

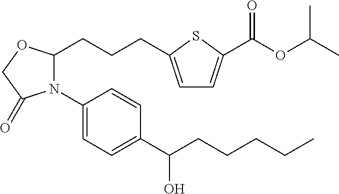

### Isopropyl 5-(3-(3-(4-(1-hydroxyhexyl)phenyl)-4-oxooxazolidin-2-yl)propyl)thiophene-2-carboxylate

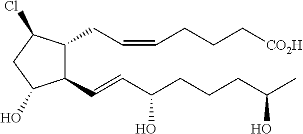

### (Z)-7-((1R,2R,3R,5R)-5-chloro-2-((3S,7R,E)-3,7-dihydroxyoct-1-enyl)-3-hydroxycyclopentyl)hept-5-enoic acid

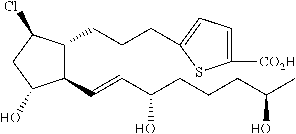

### 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid

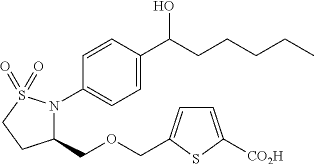

### 2-Thiophenecarboxylic acid, 5-[[[2-[4-(1-hydroxyhexyl)phenyl]-1,1-dioxido-3-isothiazolidinyl]methoxy]methyl]-

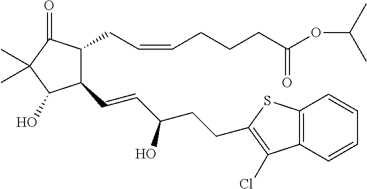

### (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate

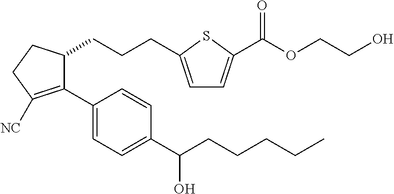

### 2-hydroxyethyl 5-(3-((1S)-3-cyano-2-(4-(1-hydroxyhexyl)phenyl)cyclopent-2-enyl)propyl)thiophene-2-carboxylate

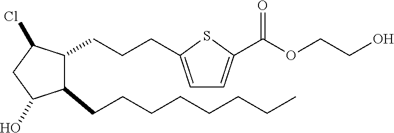

### 2-hydroxyethyl 5-(3-((1R,2R,3R,5R)-5-chloro-3-hydroxy-2-octylcyclopentyl)propyl)thiophene-2-carboxylate

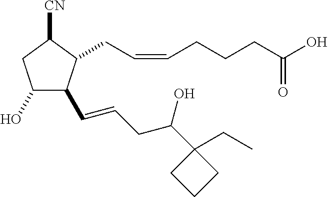

### (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid

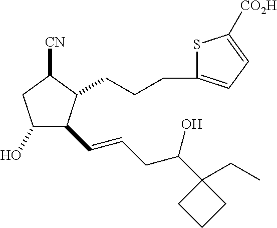

### 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid

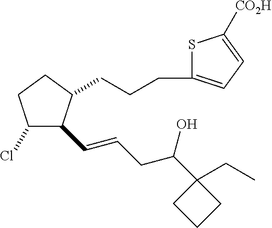

### 5-(3-((1S,2S,3R)-3-chloro-2-((E)-4-(1-ethylcyclobutyl)-4-hydroxybut-1-enyl)cyclopentyl)propyl)thiophene-2-carboxylic acid

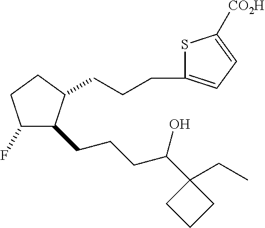

### 5-(3-((1S,2R,3R)-2-(4-(1-ethylcyclobutyl)-4-hydroxybutyl)-3-fluorocyclopentyl)propyl)thiophene-2-carboxylic acid

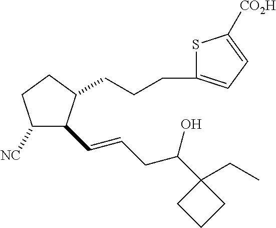

### 5-(3-((1S,2S,3R)-3-cyano-2-((E)-4-(1-ethylcyclobutyl)-4-hydroxybut-1-enyl)cyclopentyl)propyl)thiophene-2-carboxylic acid

Examples of the second therapeutically active agent include the following:

β-Blockers (or β-adrenergic antagonists) including carteolol, levobunolol, metiparanolol, timolol hemihydrate, timolol maleate, β1-selective antagonists such as betaxolol, and the like, or pharmaceutically acceptable salts or prodrugs thereof;
Adrenergic Agonists including
non-selective adrenergic agonists such as epinephrine borate, epinephrine hydrochloride, and dipivefrin, and the like, or pharmaceutically acceptable salts or prodrugs thereof; and
α₂-selective adrenergic agonists such as apraclonidine, brimonidine, and the like, or pharmaceutically acceptable salts or prodrugs thereof;
Carbonic Anhydrase Inhibitors including acetazolamide, dichlorphenamide, methazolamide, brinzolamide, dorzolamide, and the like, or pharmaceutically acceptable salts or prodrugs thereof;
Cholinergic Agonists including
direct acting cholinergic agonists such as carbachol, pilocarpine hydrochloride, pilocarbine nitrate, pilocarpine, and the like, or pharmaceutically acceptable salts or prodrugs thereof;
chlolinesterase inhibitors such as demecarium, echothiophate, physostigmine, and the like, or pharmaceutically acceptable salts or prodrugs thereof;
Glutamate Antagonists and other neuroprotective agents such as Ca²⁺ channel blockers such as memantine, amantadine, rimantadine, nitroglycerin, dextrophan, detromethorphan, CGS-19755, dihydropyridines, verapamil, emopamil, benzothiazepines, bepridil, diphenylbutylpiperidines, diphenylpiperazines, HOE 166 and related drugs, fluspirilene, eliprodil, ifenprodil, CP-101,606, tibalosine, 2309BT, and 840S, flunarizine, nicardipine, nifedimpine, nimodipine, barnidipine, verapamil, lidoflazine, prenylamine lactate, amiloride, and the like, or pharmaceutically acceptable salts or prodrugs thereof;
Prostamides such as bimatoprost, or pharmaceutically acceptable salts or prodrugs thereof; and
Prostaglandins including travoprost, UFO-21, chloprostenol, fluprostenol, 13,14-dihydro-chloprostenol, isopropyl unoprostone, latanoprost, tafluprost, and the like.
Cannabinoids including CB1 agonists such as WIN-55212-2 and CP-55940 and the like, or pharmaceutically acceptable salts or prodrugs thereof.
steroids including triamcinolone, dexamethasone, fluocinolone, and the like.

One embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

One embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of treating glaucoma comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

One embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

One embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; and fluocinolone, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and 14-dihydro-chloprostenol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and 2309BT, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and acetazolamide, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and amantadine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and amiloride, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and apraclonidine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and barnidipine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and benzothiazepines, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and bepridil, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and betaxolol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and bimatoprost, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and brimonidine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and brinzolamide, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carbachol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and carteolol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CGS-19755, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and chloprostenol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-101, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and CP-55940, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and demecarium, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and detromethorphan, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and dextrophan, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dichlorphenamide, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and dipivefrin, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and dorzolamide, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and echothiophate, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and eliprodil, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and emopamil, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine borate, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and epinephrine hydrochloride, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and flunarizine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluprostenol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and fluspirilene, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and HOE 166, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and ifenprodil, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and isopropyl unoprostone, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and latanoprost WIN-55212-2, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and levobunolol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and lidoflazine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and memantine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and methazolamide, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and metiparanolol, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and nicardipine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nifedimpine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nimodipine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and nitroglycerin, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and physostigmine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarbine nitrate, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and pilocarpine hydrochloride, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and prenylamine lactate, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and travoprost, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and rimantadine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and tibalosine, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol hemihydrate, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and timolol maleate, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and UFO-21, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and verapamil, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; and tafluprost, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and triamcinolone, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and dexamethasone, or a pharmaceutically acceptable salt thereof; to a mammal in need thereof.

Another embodiment is a method of reducing intraocular pressure comprising administering a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C, 6 alkyl ester, a phenyl ester, or a 2-hydroxyethyl ester thereof; and fluocinolone, or a pharmaceutically acceptable salt thereof; to a mammal in need thereof.

One embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C, 6 alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C, 6 alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C, 6 alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of (Z)-7-((1R,2S,3R,5R)-5-chloro-3-hydroxy-2-(4-((R)-1-hydroxyhexyl)phenyl)cyclopentyl)hept-5-enoic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-((((R)-1-(4-((S)-1-hydroxyhexyl)phenyl)-5-oxopyrrolidin-2-yl)methoxy)methyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((S)-1-(4-((S)-1-hydroxyhexyl)phenyl)-5-oxopyrrolidin-2-yl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 2-(2-((2R)-1-(4-(1-hydroxyhexyl)phenyl)-5-oxopyrrolidin-2-yl)ethylthio)thiazole-4-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-(3-(4-(1-hydroxyhexyl)phenyl)-2,5-dioxoimidazolidin-4-yl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-(3-(4-(1-hydroxyhexyl)phenyl)-4-oxooxazolidin-2-yl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of (Z)-7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((1S)-3-cyano-2-(4-(1-hydroxyhexyl)phenyl)cyclopent-2-enyl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((1R,2R,3R,5R)-5-chloro-3-hydroxy-2-octylcyclopentyl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

One embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of (Z)-isopropyl 7-((1R,2S,3R,5R)-5-chloro-3-hydroxy-2-(4-((R)-1-hydroxyhexyl)phenyl)cyclopentyl)hept-5-enoate, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of isopropyl 5-((((R)-1-(4-((S)-1-hydroxyhexyl)phenyl)-5-oxopyrrolidin-2-yl)methoxy)methyl)thiophene-2-carboxylate, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of isopropyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 2-hydroxyethyl 5-(3-((S)-1-(4-((S)-1-hydroxyhexyl)phenyl)-5-oxopyrrolidin-2-yl)propyl)thiophene-2-carboxylate, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of isopropyl 2-(2-((2R)-1-(4-(1-hydroxyhexyl)phenyl)-5-oxopyrrolidin-2-yl)ethylthio)thiazole-4-carboxylate, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((1R,2R,3R,5R)-5-chloro-2-(3,5-dichlorophenethyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-2,5-dioxoimidazolidin-4-yl)propyl)thiophene-2-carboxylate, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of isopropyl 5-(3-(3-(4-(1-hydroxyhexyl)phenyl)-4-oxooxazolidin-2-yl)propyl)thiophene-2-carboxylate, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of (Z)-7-((1R,2R,3R,5R)-5-chloro-2-((3S,7R,E)-3,7-dihydroxyoct-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((1R,2R,3R,5R)-5-chloro-2-((3S,7R,E)-3,7-dihydroxyoct-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 2-Thiophenecarboxylic acid, 5-[[[2-[4-(1-hydroxyhexyl)phenyl]-1,1-dioxido-3-isothiazolidinyl]methoxy]methyl]-, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of (Z)-isopropyl 7-((1R,4S,5R)-5-((R,E)-5-(3-chlorobenzo[b]thiophen-2-yl)-3-hydroxypent-1-enyl)-4-hydroxy-3,3-dimethyl-2-oxocyclopentyl)hept-5-enoate, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 2-hydroxyethyl 5-(3-((1S)-3-cyano-2-(4-(1-hydroxyhexyl)phenyl)cyclopent-2-enyl)propyl)thiophene-2-carboxylate, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 2-hydroxyethyl 5-(3-((1R,2R,3R,5R)-5-chloro-3-hydroxy-2-octylcyclopentyl)propyl)thiophene-2-carboxylate, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of (Z)-7-((1S,2R,3R,5R)-5-cyano-2-((E)-4-(1-ethylcyclobutyl)-4-hydroxybut-1-enyl)-3-hydroxycyclopentyl)hept-5-enoic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((1S,2R,3R,5R)-5-cyano-2-((E)-4-(1-ethylcyclobutyl)-4-hydroxybut-1-enyl)-3-hydroxycyclopentyl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((1S,2S,3R)-3-chloro-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((1S,2R,3R)-2-(4-(1-ethylcyclobutyl)-4-hydroxybutyl)-3-fluorocyclopentyl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; 14-dihydro-chloprostenol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; 2309BT, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; acetazolamide, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; amantadine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; amiloride, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; apraclonidine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; barnidipine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; benzothiazepines, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; bepridil, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; betaxolol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; bimatoprost, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; brimonidine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; brinzolamide, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; carbachol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; carteolol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; CGS-19755, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; chloprostenol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; CP-101, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; CP-55940, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; demecarium, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; detromethorphan, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dextrophan, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dichlorphenamide, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dipivefrin, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dorzolamide, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; echothiophate, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; eliprodil, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; emopamil, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine borate, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; epinephrine hydrochloride, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; flunarizine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; fluprostenol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; fluspirilene, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; HOE 166, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; ifenprodil, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; isopropyl unoprostone, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; latanoprost WIN-55212-2, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; levobunolol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; lidoflazine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; memantine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; methazolamide, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; metiparanolol, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nicardipine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nifedimpine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nimodipine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; nitroglycerin, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; physostigmine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; pilocarbine nitrate, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; pilocarpine hydrochloride, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; prenylamine lactate, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; travoprost, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; rimantadine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; tibalosine, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; timolol hemihydrate, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; timolol maleate, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; UFO-21, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; verapamil, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; tafluprost, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; triamcinolone, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; dexamethasone, or a pharmaceutically acceptable salt thereof; and a pharmaceutically acceptable excipient.

Another embodiment is a composition comprising a combination of 5-(3-((1S,2S,3R)-3-cyano-2-((E)-4-(1-ethylcyclobutyl)-4-hydroxybut-1-enyl)cyclopentyl)propyl)thiophene-2-carboxylic acid, or a pharmaceutically acceptable salt, a C₁₋₆ alkyl ester, a phenyl ester, or a 2-hydroxyethyl ester thereof; fluocinolone, or a pharmaceutically acceptable salt thereof; and a pharmaceutically acceptable excipient.

1-24. (canceled)

25. A method of treating glaucoma or ocular hypertension comprising administering a prostaglandin agonist and a second therapeutically active agent to a mammal in need thereof, wherein said second therapeutically active agent is selected from: β-Blockers, Adrenergic Agonists, non-selective adrenergic agonists, α₂-selective adrenergic agonists, Carbonic Anhydrase Inhibitors, Cholinergic Agonists, direct acting cholinergic agonists, chlolinesterase inhibitors, Glutamate Antagonists, Ca²⁺ channel blockers, Prostamides, Prostaglandins, Cannabinoids, and combinations thereof.

26. The method of claim 25 wherein the prostaglandin agonist and the second therapeutically active agent are administered in separate dosage forms.

27. The method of claim 25 wherein the prostaglandin agonist and the second therapeutically active agent are administered in a single dosage form.

28. The method of claim 25 wherein the prostaglandin agonist is a selective agonist of the prostaglandin EP2 or prostaglandin EP4 receptor.

29. The method of claim 25 wherein the prostaglandin agonist is a compound selected from: 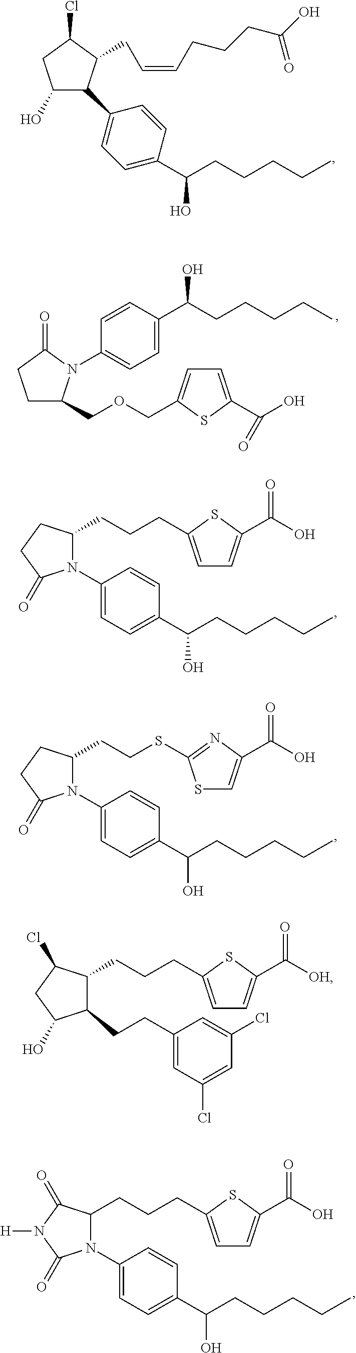 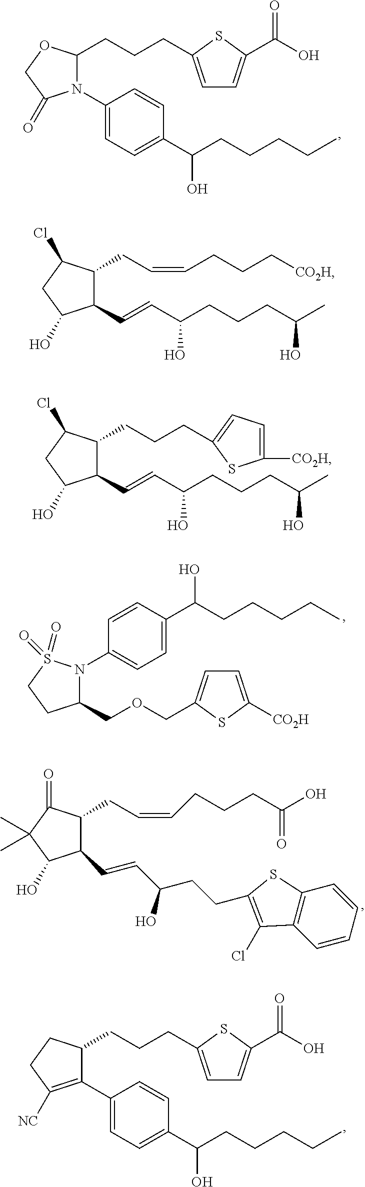 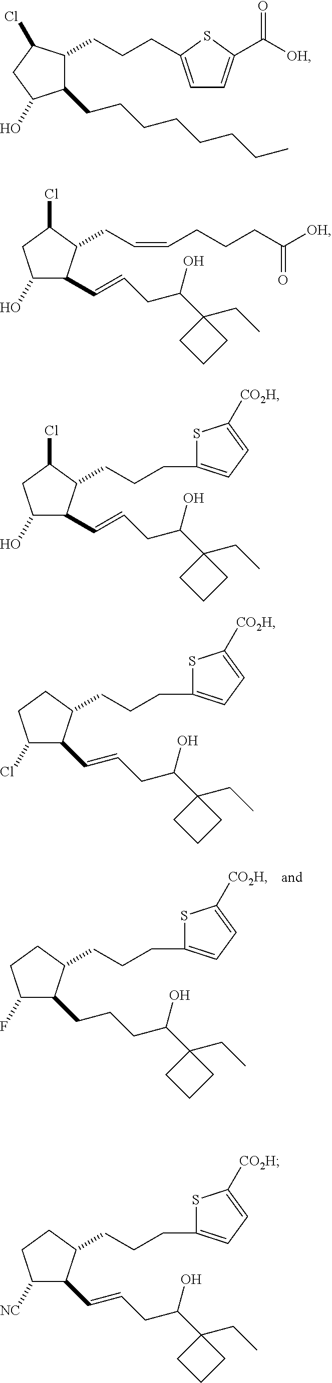
or is a C₁₋₆ alkyl ester, a phenyl ester, a 2-hydroxyethyl ester, or a pharmaceutically acceptable salt thereof.

30. The method of claim 29 wherein the prostaglandin agonist is 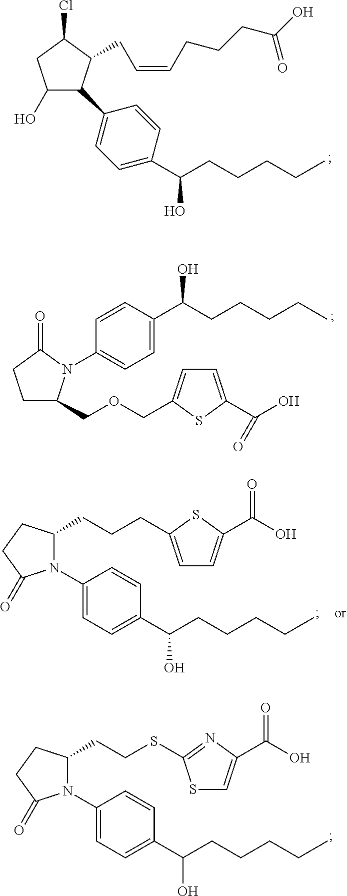
or is a salt, isopropyl ester, or a 2-hydroxyethyl ester thereof.

31. The method of claim 29 wherein the prostaglandin agonist is 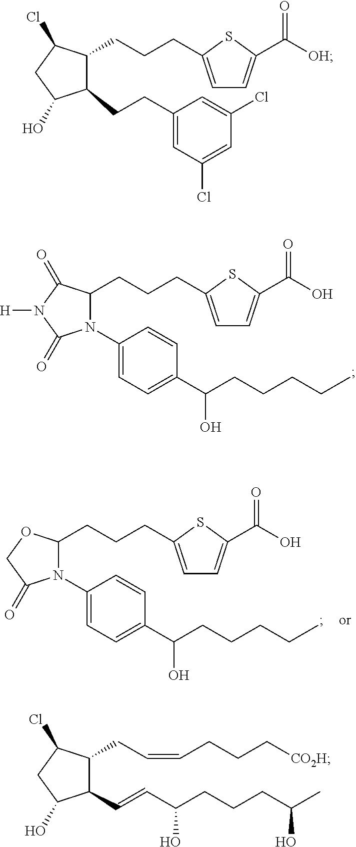
or is a salt, isopropyl ester, or a 2-hydroxyethyl ester thereof.

32. The method of claim 29 wherein the prostaglandin agonist is 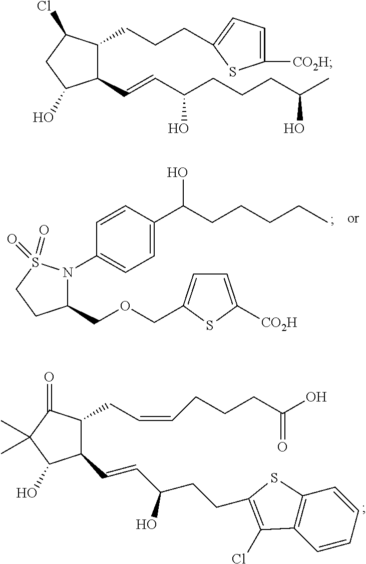
or is a salt, isopropyl ester, or a 2-hydroxyethyl ester thereof.

33. The method of claim 29 wherein the prostaglandin agonist is 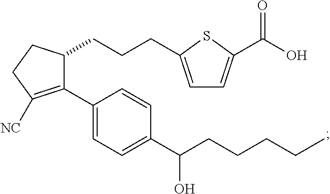
or is a salt, isopropyl ester, or a 2-hydroxyethyl ester thereof.

34. The method of claim 29 wherein the prostaglandin agonist is 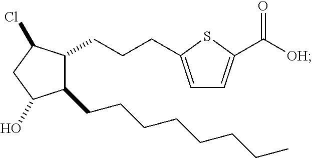
or is a salt, isopropyl ester, or a 2-hydroxyethyl ester thereof.

35. The method of claim 29 wherein the prostaglandin agonist is 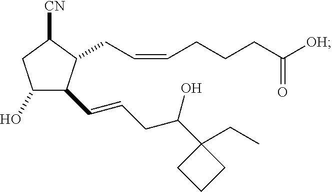
or is a salt, isopropyl ester, or a 2-hydroxyethyl ester thereof.

36. The method of claim 29 wherein the prostaglandin agonist is 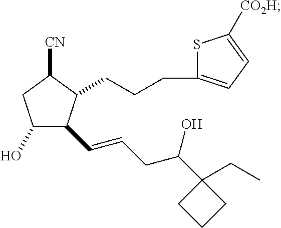
or is a salt, isopropyl ester, or a 2-hydroxyethyl ester thereof.

37. The method of claim 29 wherein the prostaglandin agonist is 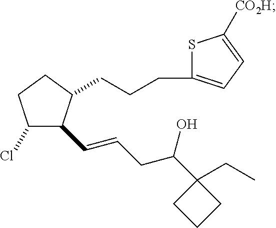
or is a salt, isopropyl ester, or a 2-hydroxyethyl ester thereof.

38. The method of claim 29 wherein the prostaglandin agonist is 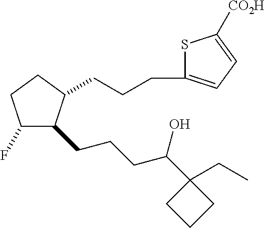
or is a salt, isopropyl ester, or a 2-hydroxyethyl ester thereof.

39. The method of claim 29 wherein the prostaglandin agonist is 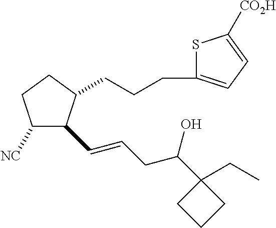
or is a salt, isopropyl ester, or a 2-hydroxyethyl ester thereof.
